(19) United States
(12) Patent Application Publication
(10) Pub. No.: US 2005/0245903 A1
(43) Pub. Date: Nov. 3, 2005

(54) Method and apparatus for controllably aspirating fluids from surgical sites

(76) Inventors: Yevgeniy Kuklin, Woodland Hills, CA (US); Norm Turoff, Poway, CA (US)

Correspondence Address: JAMES E. BRUNTON, ESQ., P. O. BOX 29000, GLENDALE, CA, 91209, US

(21) Appl. No.: 10/836,446

(22) Filed: Apr. 29, 2004

(51) Int. Cl. A61K 9/22

(52) U.S. Cl. 604/890.1

(57) ABSTRACT

A surgical aspiration system and the method of using the same for aspirating fluid from a surgical site at a desired aspiration rate. The system includes an interface component for providing an interface with the surgical site; a variable rate pump operably associated with the interface component for controllably generating a vacuum at the surgical site to cause a flow of fluids from the surgical site; a collector chamber interconnected with the interface component and with the pump for collecting liquids flowing from the surgical site; and a control system operably associated with the pump for controlling the pumping rate thereof in a manner to maintain the desired aspiration rate at the surgical site, the control system including a sensor for sensing a change in vacuum at the interface component due to liquid entering the interface component and for controlling the pumping rate of the pump in response thereto.

### BACKGROUND OF THE INVENTION

1. Field of the Invention

The present invention relates generally to methods and apparatus for controlling fluid flow. More particularly, the invention concerns methods and apparatus for controllably aspirating fluids from surgical sites such as the human eye during various surgical procedures.

2. Description of the Prior Art

Great strides have been made in recent years in all types of surgical procedures and particularly in intraocular surgery where a number of novel surgical instruments for use in ocular surgery have been developed.

In connection with these advances a number of patents have been issued concerning various types of apparatus for automatically controlling the cutting and sectioning of materials from various types of surgical zones including ocular surgery sites. In ocular surgery, it is of course vitally important that fluids and various types of debris be removed from the ocular site without doing damage to the eye. In this regard, a number of ophthalmologic fluid control systems have been suggested for surgical irrigation and aspiration. One such system is disclosed in U.S. Pat. No. 4,935,005 issued to Haines. This Patent discloses a fluid flow control apparatus especially adapted for use with an ultrasonic surgical tool, which provides for irrigation of a surgical site and for aspirating fluid from the site.

Another prior art apparatus for automatically regulating and controlling the vacuum level at which unwanted material is suctioned from a surgical zone is disclosed in U.S. Pat. No. 4,395,258 issued to Wang et al. The Wang et al. apparatus incorporates a pressure transducer, which continuously senses the vacuum level of a debris receptacle when suctioning of material from the surgical zone into the receptacle is desired.

The Prior Art Patent to Kelman No. U.S. Pat. No. 4,465,470 discloses an apparatus for irrigating and aspirating the eye utilizing valves in a conduit and conduit branches to control the flow of fluid into and out of the eye under the force of gravity. In a similar vein, U.S. Pat. No. 5,810,765 issued to Oda discloses an irrigation/aspiration apparatus for use in a cataractal operation. The Oda apparatus comprises an irrigation fluid supply means for supplying an irrigation fluid contained in an irrigation bottle to the patient's eye and an irrigation fluid aspirating means for aspirating, together with loosened tissues and the like, the irrigation fluid supplied into the patient's eye. The irrigation/aspiration apparatus also includes an irrigation flow rate varying means for varying the irrigation rate of the irrigation fluid at which the irrigation fluid supply means supplies the irrigation fluid. Further, the Oda apparatus includes and an irrigation flow rate control means for controlling the irrigation flow rate varying means according to the operating condition of the irrigation fluid aspirating means to regulate the irrigation flow rate so that the variation of the pressure in the anterior chamber of the patient's eye is suppressed during the cataractal operation.

A more recent Patent issued to Neubert No. U.S. Pat. No. 6,599,277 discloses an opthamathic surgical pump system that includes a collection reservoir and venturi pump for creating a vacuum level for aspirating surgical tissue from a surgical site to a collection reservoir. Aspirating tubing connects to each of the surgical hand piece and the collection reservoir. A flow meter is electrically connected to either the aspiration tube or the collection reservoir. Control circuitry is connected to the flow meter and to the venturi pump for carrying a vacuum level of the pump to maintain a desired flow rate of the fluid and tissue being aspirated from the surgical site.

U.S. Pat. No. 5,971,977 issued to Korenfeld entitled Surgical Laser Smoke Plume Evacuator discloses the use of an aspirating pump in conjuction with a speculum for removing smoke from the eye of the patient during laser surgery. Similarly, U.S. Pat. No. 5,762,608 issued to Minnich is directed toward a combined eyelid retractor and eyelid-flushing device.

As will be discussed in greater detail hereinafter a primary object of the present invention is to provide an apparatus for aspirating fluids from surgical sites, such as the human eye at a precise, predetermined rate during various types of surgical procedures. The apparatus of one form of the invention comprises a diaphragm pump, which is operably interconnected with a conventional aspirating speculum via a fluid conduit and a collection chamber. Disposed between the speculum and the diaphragm pump is a pressure sensor which senses pressure change during use. When the diaphragm pump is initially actuated, fluid, which is primarily air, will flow from the speculum through the connecting conduit to the diaphragm pump. However at such time as the fluid flowing from the eye comprises both air and water the pressure in the conduit will drop. This pressure drop is sensed by the pressure sensor of the apparatus, which automatically causes the diaphragm pump to pump at a greater rate so that fluid flowing through the fluid conduit will increase substantially from its initial aspiration level. When the sensor senses that substantially all of the liquid has been pumped from the surface of the eye, the diaphragm pump will automatically switch back to the initial vacuum level. A bacterial filter is provided at the outlet port of the collection chamber. The filter acts as an effective bacterial filter and also provides viable overflow protection under conditions of maximum possible pressure drop.

### SUMMARY OF THE INVENTION

By way of brief summary, the present invention concerns a novel surgical aspiration system for aspirating fluid from a surgical site at a controllable aspiration rate. In one form of the invention, the system comprises interface means for providing an interface with the surgical site; a variable rate pumping means operably associated with the interface means for controllably generating a vacuum at the interface means to cause a flow of fluids from the surgical site; a collector chamber interconnected with the interface means and with the pumping means for collecting liquids flowing from the surgical site; and novel control means operably associated with the pumping means for controlling the pumping rate thereof in a manner to maintain a substantially constant vacuum at the interface means, the control means including sensor means for sensing a change in vacuum at the interface means and for controlling the pumping rate of the pumping means in response thereto.

In light of the foregoing, it is an object of the present invention to provide a method and apparatus for controllably aspirating fluids from surgical sites, including ocular sites during surgery. More particularly, it is an object of the invention to provide an apparatus for precisely controlling the vacuum level at the surgical site.

Another object of the invention is to provide an apparatus of the aforementioned character, which includes collection means operably associated with the interface means for collecting liquids extracted from the surgical site.

Another object of the invention is to provide an apparatus of the character described which includes filter means disposed intermediate the collection means and the source of vacuum for effectively filtering bacteria from the fluids flowing toward the source of vacuum.

Another object of the invention is to provide an apparatus as described in the preceding paragraph, in which the filter means also provides viable overflow protection under conditions of maximum possible pressure drop.

Another object of the invention is to provide an apparatus comprising control means, which includes a controller component and a sensor component for continually sensing a change in vacuum level at the interface means caused by liquid being extracted from the surgical site.

Another object of the invention is to provide an apparatus of the character described in the preceding paragraph in which the sensor component, upon sensing a change in vacuum level within the collector chamber due to liquid being extracted from the surgical site, generates and transmits to the pump, which is the source of vacuum, a signal which will cause an increase in the pumping rate of the pump.

Another object of the invention is to provide valve means for venting the system at the completion of the surgical procedure.

Another object of the invention is to provide an apparatus of the type described in the preceding paragraphs which is highly reliable in operation is easy-to-use with a minimum of training.

Another object of the invention is to provide an apparatus of the character described in which the operating components are housed within a compact protective housing and in which the apparatus is of a simple design which can be inexpensively manufactured and maintained.

The foregoing, as well as other objects of the invention, will be achieved by the apparatus of the invention, the details of which are described in the paragraphs, which follow.

### BRIEF DESCRIPTION OF THE DRAWINGS

FIG. 1 is a generally perspective view of one form of the apparatus of the present invention for controllably aspirating fluids from the surface of the human eye during ocular surgery.

FIG. 2 is a generally schematic view of the form of the apparatus shown in FIG. 1.

FIG. 3 is a generally perspective view of one form of an aspirating speculum that can be used in connection with the apparatus of the invention.

FIG. 4 is a generally graphical representation showing pressure as a function of time.

### DESCRIPTION OF THE INVENTION

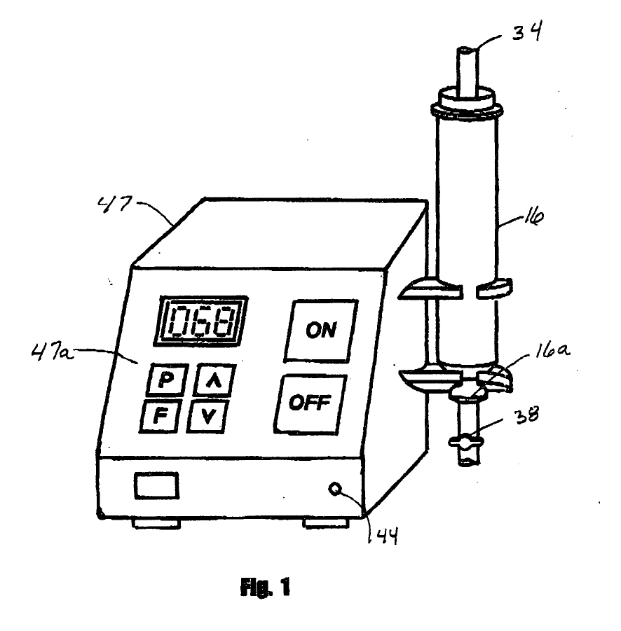

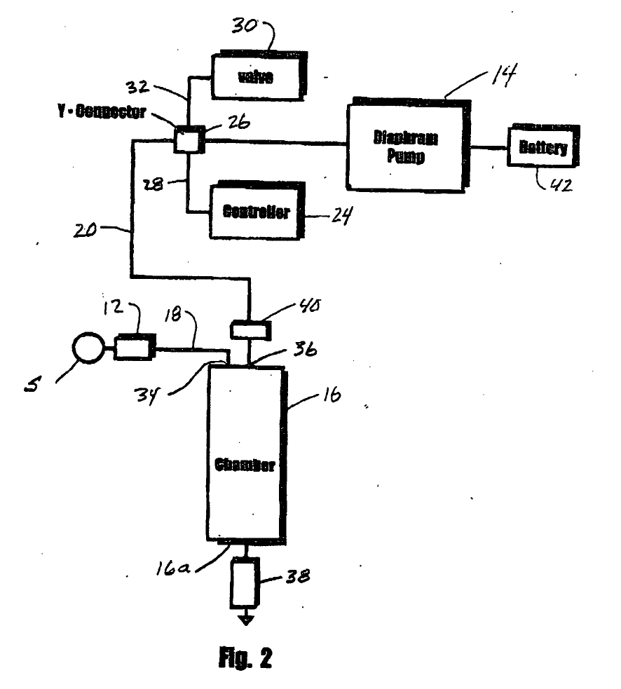

Referring to the drawings and particularly to FIGS. 1 and 2, one form of the system of the present invention for controllably aspirating fluids from a surgical site “S” is there shown. The system here comprises interface means, generally designated by the 12, for providing an interface with the surgical site. An aspiration vacuum source, such as a variable rate pumping means as, by way of example, a diaphragm pump 14, is provided to generate an aspirating vacuum at the interface means to controllably withdraw fluid from the surgical site fluid, including gases and liquid.

A collector chamber 16 is operably associated with pump 14 for collecting the liquid component of the fluid flowing from the surgical site toward the pump. A first fluid flow conduit 18 interconnects the interface means with the collector chamber and a second fluid flow conduit 20 interconnects the connector chamber with pump 14.

An important feature of the system of the invention is a novel control means that is operably associated with pump 14 for controlling the pumping rate of the pump in a manner to maintain a desired aspiration rate at the interface means. In the present form of the invention this important control means comprises a controller and a sensor means for sensing a change in vacuum at the interface means and for generating and transmitting a signal to the controller which in turn transmits a signal to the pump to control the pumping rate thereof. The details of this important control means will presently be described.

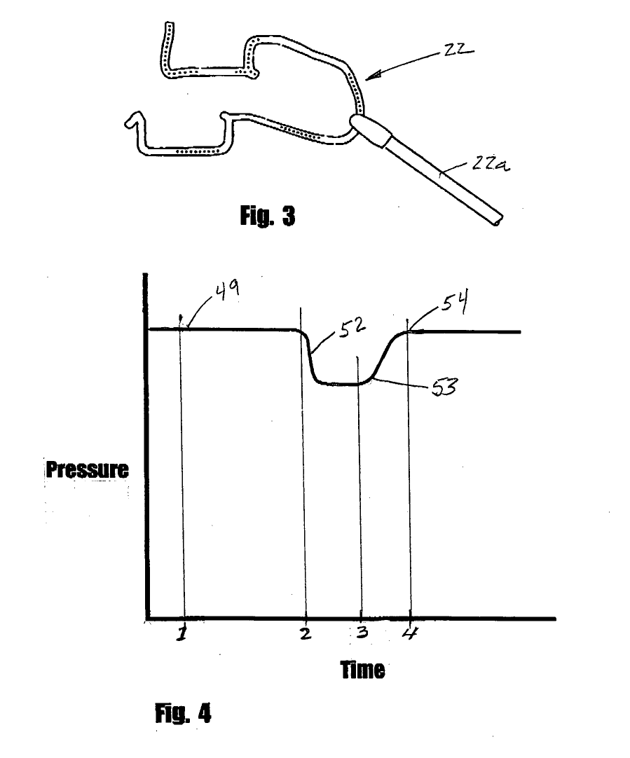

The apparatus of the invention can be used in connection with various types of surgical procedures and with various types of surgical sites, including ophthalmic surgical sites. When used in connection with ophthalmic surgery, the interface means comprises a conventional aspirating ocular speculum 22, such as that shown in FIG. 3 of the drawings. When used in connection with other types of surgical procedures, the interface means can comprise various types of vacuum tips of suitable configuration. Aspirating ocular speculum 22 is readily commercially available from a number of sources, including Katena Products, Inc., of Denville, N.J. Similarly, aspiration pump 14 is readily commercially available from several sources, including KNF Neuberger of Trenton, N.J. The aspiration pump is preferably a microprocessor controlled diaphragm pump which is capable of achieving a maximum flow rate of about 6 liters per minute and a vacuum level of about 100 millimeters of mercury (mmHg).

In the present form of the invention, the control means 24 comprises a controller component as well as a sensor component, or sensor means for sensing a change in vacuum at the interface means and for generating and transmitting a signal to pump 14 to control the pumping rate thereof. The sensor means of the system here comprises a semi conductor pressure sensor of the type commercially available from a number of sources, including Omega Engineering of Stamford, Conn.

The previously identified first and second flow conduits 18 and 20 can take several forms, but preferably are constructed from plastic tubing of the type commercially available from the Qosina Company of Edgewood, N.Y. As illustrated in FIG. 2, a standard, commercially available “Y” connector 26 is interconnected with second flow conduit 20 intermediate its ends. Controller means 24 is interconnected with one leg of “Y” connector 26 by means of flow conduit 28 and a valve means, here provided as a control valve 30, is interconnected with another leg of the “Y” connector by means of a flow conduit 32. The function of control valve 30 will presently be described.

Collection chamber 16 here comprises an approximately 64 cubic centimeter (cc), rigid polyvinyl chlorine (PVC) chamber having inlet and outlet ports 34 and 36 respectively. A stopcock 38 is provided at the lower end of chamber 16 to drain liquid from the chamber for appropriate disposal. Interconnected with second fluid flow conduit 20 at a location intermediate the outlet 36 of the collector chamber 16 and “Y” connector 26 is filter means for filtering bacteria from fluid flowing through the second fluid flow conduit. This filter means is here provided in the form of a commercially available bacterial filter 40 having a pore size of about 0.3 microns. In addition to acting as a bacterial filter, filter 40 uniquely functions as a collection chamber overflow protector and provides satisfactory overflow protection at maximum possible pressure drop of 760 mmHg on the filter for at least 8 hours of continuous operation.

For reasons of safety, in the present embodiment of the invention the aspiration pump 14 is powered by a battery 42 which ensures at least 8 hours of continuous operation. Battery charge is controlled by a conventional microprocessor (not shown) and charge level is displayed on the front panel by an indicator 44 (FIG. 1).

As indicated in FIG. 1 of the drawings, pump 14 as well as the controller means 24 are housed within a compact housing 47 having a front display panel 47a. Controller means 24 includes a programmed microprocessor (not separately shown), which responds to user controls provided on the front display panel, such as pressure control “P” and a flow rate control “F”. Controller means 24 also responds to changes in the vacuum level within the collector chamber 16 as measured by the sensor means of the controller means and adjusts the pumping rate of pump 14 as required to maintain a desired aspiration rate.

In accordance with one form of the method of the invention, the ocular speculum 22 is first secured to the patient in a conventional manner so as to permit the upper eyelid to be retracted during the performance of the surgical procedure. The first fluid flow conduit 18 is then interconnected with the handle 22a of the ocular speculum in a conventional manner. Next, using the “ON” switch provided on housing face 47a, the aspirating pump 14 is appropriately energized (FIG. 1). In this regard, it is to be noted that the aspiration pump 14 is capable of being operated in two modes, namely a vacuum control mode and flow control mode. In the vacuum control mode the diaphragm pump maintains a predetermined, operator set vacuum level or negative pressure in the collection chamber. During pump operation, the pressure sensor continuously monitors the vacuum level in the collection chamber and in first conduit 18 and provides feedback to the controller component of the controller means which controls the pumping rate of pump 14 in a manner to maintain the aspiration rate at the interface means within a predetermined range. The flow control mode of the aspiration pump is here specially designed for use with ophthalmic aspiration speculum 22 to controllably evacuate fluids from the surface of an eye during various types of vision correction procedures.

As indicated in FIG. 4, which plots pressure against time, when first energized at time “1”, pump 14 functions in a manner to achieve minimum possible air flow through the aspirating speculum by producing an initial vacuum level designated by the numeral 49, which, by way of example, is about 750 millimeters of mercury (mmHg). However, at time “2” during the surgery, when liquid first reaches the inlet apertures of the speculum, resistance to the fluid flow, of course, changes. At this point, as illustrated in FIG. 4, the sensor means senses a change in vacuum level in first conduit 18 and in collector chamber 16 both of which are in the path of fluid aspiration from the surgical site. In response to this change in pressure, identified by the numeral 52 in FIG. 4, the sensor means generates and transmits an appropriate signal to the controller component of the controller means 24. The controller microprocessor of the controller responds to this signal by appropriately increasing the speed of the aspiration pump 14, with which it is operably associated, to increase the vacuum level at the interface means to achieve the desired aspiration rate for liquid extraction as indicated at time 3 in FIG. 4. Continual monitoring of the vacuum level within the collection chamber will determine the point at which all of the liquid has been removed from the surgical site as indicted by the numeral 53 in FIG. 4. At this point, the controller will appropriately adjust the pumping speed of pump 14 to once again achieve the normal initial low flow operation. Upon completion of the surgical procedure, pump 14 is deenergized and valve 30 is opened permitting air to flow into second conduit 32 so as to equalize pressure within the system.

In accordance with an alternate method of the invention, the pumping rate of the aspiration pump is periodically changed and changes in pressure as a result thereof are continuously monitored by the sensor means. At such time as the sensor means senses a greater than usual change in pressure drop, indicating that no more liquids are being evacuated from the surgical site, a signal is generated and transmitted to the controller. The controller, in turn, then transmits a signal to the pump causing deenergization of the pump.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

1. A surgical aspiration system for aspirating fluid from a surgical site comprising:
(a) interface means for providing an interface with the surgical site;
(b) a variable rate pumping means operably associated with said interface means for generating a vacuum at said interface means to cause a desired aspiration rate of fluids from the surgical site;
(c) a collector chamber interconnected with said interface means and with said pumping means for collecting liquids flowing from away from said surgical site; and
(d) control means operably associated with said pumping means for controlling the pumping rate thereof in a manner to maintain said desired aspiration rate at said interface means, said control means including sensor means for sensing a change in pressure at said interface means.

2. The surgical aspiration system as defined in claim 1 in which said interface means comprises a vacuum tip for aspirating fluids from the surgical site.

3. The surgical aspiration system as defined in claim 1 in which the surgical site comprises a human eye and in which said interface means comprises an aspirating ocular speculum.

4. The surgical aspiration system as defined in claim 1 in which said pumping means comprises a diaphragm pump.

5. The surgical aspiration system as defined in claim 1 further including a first fluid flow conduit interconnecting said interface means with said collector chamber and a second fluid flow conduit interconnecting said collector chamber with said pumping means, said sensor means being operably interconnected with said second fluid flow conduit.

6. The surgical aspiration system as defined in claim 5, further including filter means disposed within said second fluid flow conduit for filtering bacteria from said fluid flowing through said second conduit.

7. The surgical aspiration system as defined in claim 5, further including valve means interconnected with said second fluid flow conduit for venting fluids to atmosphere.

8. The surgical aspiration system as defined in claim 5 in which said sensor means senses the drop in pressure in said second fluid flow conduit caused by liquid flowing through said first fluid flow conduit toward said collection chamber.

9. The surgical aspiration system as defined in claim 9 in which said sensor means, upon sensing a drop in pressure in said second fluid flow conduit, generates a signal and transmits said signal to said pumping means to vary the pumping rate thereof.

10. A surgical aspiration system for aspirating fluid from a surgical site comprising:
(a) interface means for providing an interface with the surgical site;
(b) a variable rate pumping means operably associated with said interface means for generating a vacuum at said interface means to cause a desired aspiration rate of fluid from the surgical site;
(c) a collector chamber operably associated with said pumping means for collecting liquid flowing from said interface means toward said pumping means;
(d) a first fluid flow conduit interconnecting said interface means with said collector chamber;
(e) a second fluid flow conduit interconnecting said connector chamber with said pumping means; and
(f) control means operably associated with said pumping means for controlling the pumping rate thereof in a manner to maintain said desired aspiration rate of said interface means by controlling the pumping rate at said pumping means, said control means comprising sensor means for sensing a change in pressure at said interface means and for generating and transmitting a signal to said pumping means to control the pumping rate thereof

11. The surgical aspiration system as defined in claim 10, further including filter means disposed within said second fluid flow conduit for filtering bacteria from said fluid flowing through said second conduit.

12. The surgical aspiration system as defined in claim 10, further including valve means interconnected with said second fluid flow conduit for venting fluids to atmosphere.

13. The surgical aspiration system as defined in claim 10 in which said sensor means senses the drop in pressure in said second fluid flow conduit caused by liquid flowing through said first fluid flow conduit toward said collection chamber.

14. The surgical aspiration system as defined in claim 13 in which said sensor means, upon sensing a drop in pressure in said second fluid flow conduit, generates a signal and transmits said signal to said pumping means to vary the pumping rate thereof.

15. A surgical aspiration system for aspirating fluid from a surgical site comprising:
(a) interface means for providing an interface with the surgical site;
(b) a variable rate pumping means operably associated with said interface means for generating a vacuum at said interface means to cause a desired aspiration rate of fluid from the surgical site;
(c) a collector chamber operably associated with said pumping means for collecting liquid flowing from said interface means toward said pumping means;
(d) a first fluid flow conduit interconnecting said interface means with said collector chamber;
(e) a second fluid flow conduit interconnecting said connector chamber with said pumping means;
(f) filter means disposed within said second fluid flow conduit for filtering bacteria from said fluid flowing through said second conduit;
(g) valve means interconnected with said second fluid flow conduit for venting fluids to atmosphere; and
(h) control means operably associated with said pumping means for controlling the pumping rate thereof in a manner to maintain said desired aspiration rate at said interface means by controlling the pumping rate of said pumping means, said control means comprising sensor means operably associated with said second fluid flow conduit for sensing a change in pressure at said interface means caused by liquid flowing from said surgical site and for generating and transmitting a signal to said pumping means to control the pumping rate thereof.

16. A method for withdrawing fluids, including gases and liquids, from a surgical site through use of an apparatus comprising interface means for providing an interface with the surgical site; a vacuum pump operably interconnected with the interface means for creating a vacuum at the surgical site to achieve a desired aspiration rate of fluid from the surgical site; and controller means comprising a controller and sensor means for sensing a change in pressure at the interface means, for generating a signal in response thereto and for transmitting a signal to the controller, which is operably associated with the vacuum pump, to cause a change in the pumping rate thereof, the method comprising the steps of:
(a) determining an initial vacuum level at the interface means sufficient to with draw fluids from the surgical site;
(b) energizing the vacuum pump to create said initial vacuum level at the interface means;
(c) using the sensor means of the controller means, continuously monitoring the vacuum level at the interface means to determine a change in the vacuum level at the interface means as a result of liquids entering the interface means and, in response thereto, generating and transmitting a signal to the controller; and
(d) using the controller, transmitting a signal to the vacuum pump to cause an increase the pumping rate thereof sufficient to achieve the desired aspiration rate of fluids from the surgical site.

17. The method as defined in claim 16, including the further steps of:
(a) using the sensor means of the controller means, continuously monitoring the vacuum level at the surgical site to determine a change in the vacuum level at the interface means as a result of only gases entering the interface means;
and
(b) using the controller of the controller means, transmitting a signal to the vacuum pump to deenergize the vacuum pump.

18. A method for withdrawing fluids, including gases and liquids from a surgical site through use of an apparatus comprising interface means for providing in interface with the surgical site, a vacuum pump operably interconnected with the interface means for creating a vacuum sufficient to withdraw fluids from the surgical site at a desired rate; a collection chamber for collecting liquids withdrawn from the surgical site; filter means disposed between the collection chamber and the vacuum pump for filtering bacteria from fluids flowing toward the vacuum pump; and controller means comprising a controller and sensor means for sensing a change in vacuum level at the interface means, for generating a signal in response thereto and for transmitting the signal to the controller to cause the controller to change the pumping rate of the vacuum pump, the method comprising the steps of:
(a) determining an initial vacuum level at the surgical site;
(b) energizing the vacuum pump to create said initial vacuum level at the interface means;
(c) using the sensor means, continuously monitoring the vacuum level within the collection chamber to determine a change in the vacuum level therein as a result of liquids entering the interface means and, in response thereto, generating and transmitting a signal to the controller; and
(d) using the controller, transmitting a signal to the vacuum pump to cause an increase the pumping rate thereof sufficient to maintain the withdrawal of fluids fro the surgical site at the desired rate.

19. The method as defined in claim 18, including the further step of using said filter means to filter bacteria from the fluids flowing toward said vacuum pump.

20. The method as defined in claim 19, including the further steps of:
(a) using the sensor means, continuously monitoring the vacuum level within the collection chamber to determine a change in the vacuum level therein as a result of only gases entering the collection chamber, generating a signal in response thereto and transmitting said signal to said controller; and
(b) using the controller, transmitting a signal to the vacuum pump to deenergize the vacuum pump.

21. A method for withdrawing fluids, including gases and liquids from a surgical site through use of an apparatus comprising interface means for providing in interface with the surgical site, a vacuum pump operably interconnected with the interface means for creating a vacuum sufficient to withdraw fluids from the surgical site at a desired rate; a collection chamber for collecting liquids withdrawn from the surgical site; filter means disposed between the collection chamber and the vacuum pump for filtering bacteria from fluids flowing toward the vacuum pump; and controller means comprising a controller and sensor means for sensing changes in the vacuum level at the interface means, for generating a signal in response thereto and for transmitting the signal to the controller to cause the controller to change the pumping rate of the vacuum pump, the method comprising the steps of:
(a) determining an initial vacuum level at the surgical site;
(b) energizing the vacuum pump to create said initial vacuum level at the interface means;
(c) periodically changing the pumping rate of the vacuum pump;
(d) using the sensor means, continuously monitoring the vacuum level at the interface means during the periodic changes in the pumping rate of the vacuum pump to determine a change in the vacuum level indicating that only gases are entering the interface means and, in response thereto, generating and transmitting a signal to the controller; and
(e) using the controller, transmitting a signal to the vacuum pump to deenergize the vacuum pump.
